(19) United States
(12) Patent Application Publication
(10) Pub. No.: US 2022/0192290 A1
(43) Pub. Date: Jun. 23, 2022

(54) MASK WITH FACE CONTACT FUNCTION

(71) Applicant: NANOREX CO., LTD., Gyeonggi-do (KR)

(72) Inventors: Chan Bong LEE, Gyeonggi-do (KR); Jeong Ae LEE, Gyeonggi-do (KR); Ye Ji LEE, Gyeonggi-do (KR)

(21) Appl. No.: 17/127,254

(22) Filed: Dec. 18, 2020

(51) Int. Cl. A41D 13/11

(52) U.S. Cl. CPC A41D 13/1161; A61M 16/16; A41D 2400/32; A41D 13/1115

(57) ABSTRACT

A face contact function includes a contact member comprising a first cutting line provided in the upper left end of the mask body to form a first fastening protrusion by an external force, a first fastening protrusion cutting line provided in the lower left end of the mask body and providing a space in which the first fastening protrusion is to be fitted so as to maintain a state that the upper end is in close contact with the lower end, a second cutting line provided in the upper right end of the mask body to form a second fastening protrusion by an external force, and a second fastening protrusion cutting line provided in the lower right end of the mask body and providing a space in which the second fastening protrusion is to be fitted.

### BACKGROUND

The present invention relates to a mask capable of continuously filtering air inhaled through wearer's nose and mouth while stably covering the nose and mouth, and more particularly, to a mask with a face contact function that allows a wearer to directly change a structure of a mask body so that the mask body is not separated from the face regardless of a face structure and a face size of the wearer.

Earrings of a typical mask hang on the wearer's ears to provide a function of preventing a mask body portion from being separated from the wearer's face.

FIG. 1 is an exploded perspective view showing a conventional mask. Referring to FIG. 1, a mask 10 according to the conventional art includes a mask body, and earrings 15 provided on both sides of the mask body and hang on the ears.

The mask body includes a body portion 11 forming one or more layers in which a plurality of wrinkles 14 are formed, an edge portion 12 that is thermally bonded and provided on an upper end and a lower end of the body portion 11 through heat fusion, and a finishing portion 13 that is thermally fused through ultrasonic waves and provided on both sides of the body portion 11.

The edge portion 12 is thermally fused in a grounded state and coupled to the body portion 11. A wire 17 of a metal material may be inserted into the edge portion 12 so as not to be exposed to the outside. Such a wire 17 is for improving the dustproof effect by improving adhesion when wearing the mask 10, and is made of a plastic material such as metal capable of free bending so as to be in close contact with a curve of the nose part.

FIG. 2 is a side view showing a conventional mask worn by the wearer.

As shown in FIG. 2, because a lifting phenomenon occurs in the finishing portion 13 of the mask 10 in contact with a curved contour part of the wearer's cheek, fine dust or various harmful bacteria may be introduced into the respiratory organ through the mouth and nose of the wearer of the mask, and in this case, there was a problem that the effect of wearing the mask is not properly achieved.

Depending on the size of the face of the wearer wearing the mask, because the length of the earrings is short, and the mask body portion presses the wearer's face, which makes it uncomfortable to wear, or since the length of the earrings is long, the mask body portion flows down from the wearer's face and is not in close contact with the wearer's face, which causes a phenomenon in which external air directly flows into the wearer's nose and mouth.

Meanwhile, recently, with coronavirus infection-19 (COVID-19) outbreaks around the world, many masks are used and then thrown away even in a day, causing environmental pollution. In particular, in the case of earrings, although the earrings can be recycled, the earrings are thrown away, and there is room for improvement so that the earrings can play various roles, but the earrings are used only in the form of a closed curve connected to the mask, and there is also a problem such as dying of animals such as birds tied to the earrings.

### SUMMARY OF THE INVENTION

Accordingly, the present invention has been made in view of the above-mentioned problems occurring in the prior art, and it is an object of the present invention to provide a mask capable of stably filtering fine dust or various harmful bacteria contained in the air by suppressing the occurrence of a phenomenon in which a mask body in contact with the curved contour part of the cheek according to the size or curve of the wearer's face is separated, and providing various functions as well as minimizing adverse effects on the environment.

According to the present invention, without the use of a separate member such as Velcro, a state of being in close contact with the wearer's cheek is stably maintained regardless of the size or a curve of the wearer's face, or a movement of the wearer.

In addition, because there is no need to mount a member such as Velcro, etc. on a mask body, the production can be increased compared to a mask equipped with Velcro, etc., and the manufacturing cost can be reduced. In addition, the mask of the present invention can be worn for a long time because the weight of the mask is minimized.

In addition, because the mask of the present invention can maintain a state of being in close contact with the face regardless of the movement of the wearer, the effect of blocking contaminants including viruses is excellent.

In addition, it is possible to increase the utilization efficiency of the mask by giving various functions to earrings, and it is possible to prevent damage from animals such as birds being tied to the earrings, etc. by using the detachable earrings.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is an exploded perspective view showing a conventional mask.

FIG. 2 is a side view showing a conventional mask worn by the wearer.

FIG. 3 is a front view showing a mask according to an embodiment of the present invention.

FIG. 4 is a perspective view showing a mask in which a fastening protrusion of FIG. 3 is inserted into a cutting line.

FIG. 5 is a schematic view showing the mask of FIG. 3 worn by a wearer.

FIG. 6 is a perspective view showing a mask according to another embodiment of the present invention.

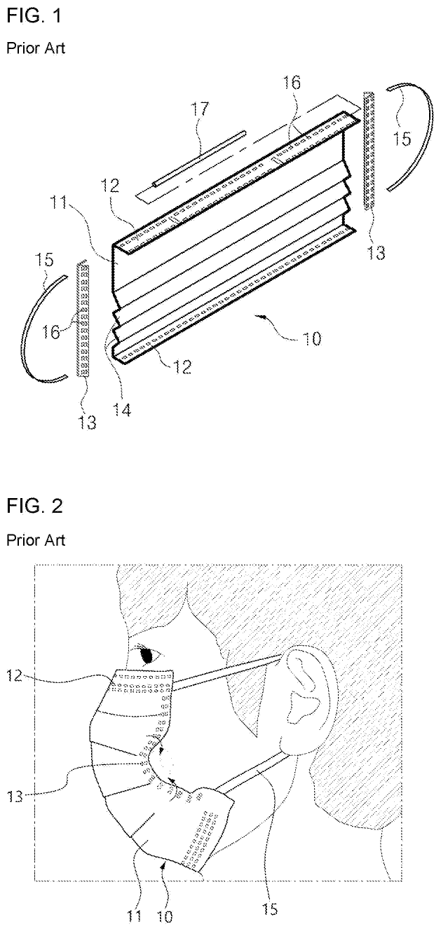

### DETAILED DESCRIPTION OF THE PREFERRED EMBODIMENTS

Hereinafter, a mask with a face contact function (hereinafter abbreviated as the ‘mask’) according to embodiments of the present invention will be described in detail with reference to the accompanying drawings.

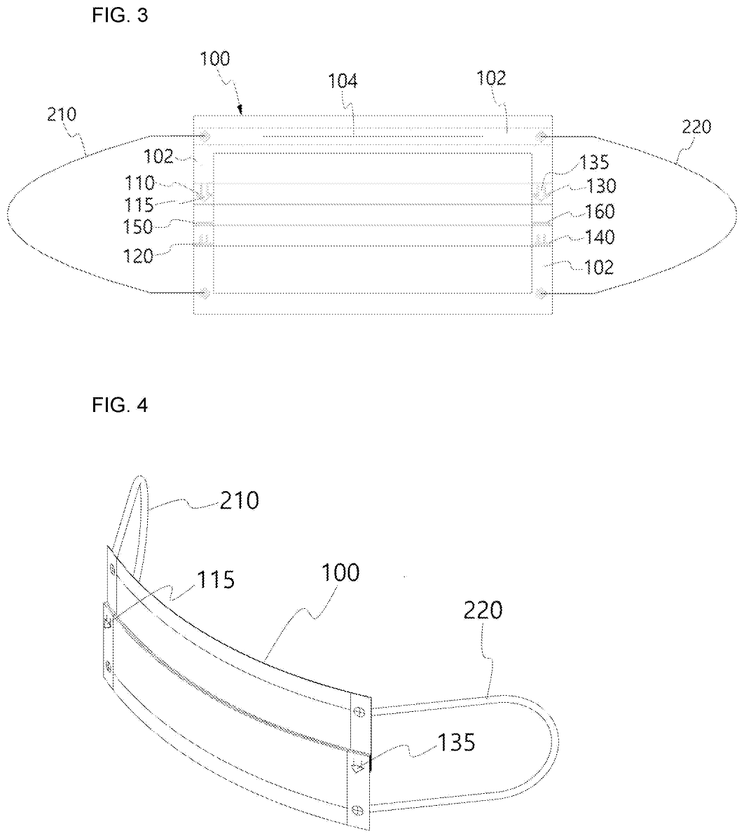

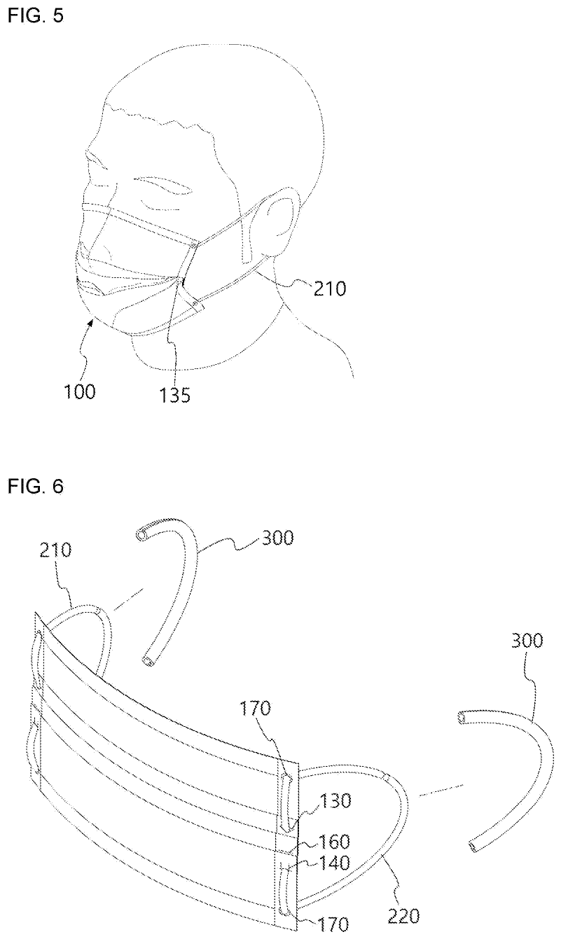

FIG. 3 is a front view showing a mask according to an embodiment of the present invention. FIG. 4 is a perspective view showing a mask in which a fastening protrusion of FIG. 3 is inserted into a cutting line. FIG. 5 is a schematic view showing the mask of FIG. 3 worn by a wearer.

Referring to FIGS. 3 to 5, the mask according to the present invention includes a mask body 100 including a contact member, and earrings 200 respectively provided on the left and right sides of the mask body 100.

At this time, the contact member is preferably provided on a bonding surface 102 of the mask body 100 so as not to participate in the movement of the air during a breathing process of the wearer. The bonding surface 102 means a part in which a bonding line is formed along the edge of the mask body 100 through thermal fusion or ultrasonic fusion so that several filters constituting the mask body 100 are coupled to each other.

Hereinafter, each component will be described in more detail with reference to the drawings.

Referring to FIGS. 3 to 5, the mask according to the present invention includes the mask body 100.

The mask body 100 is seated on the wearer's face so as to cover the wearer's nose and mouth, and filters harmful substances such as fine dust contained in the air introduced from the outside direction to introduce only clean air into the interior.

To this end, the mask body 100 may be configured such that two or more of cotton, synthetic resin, non-woven fabric filter, synthetic resin filter, and electrostatic filter are stacked individually or in combination.

Here, the electrostatic filter means a filter that is electrostatically treated using an ultra-high voltage current, and static electricity may capture fine dust that has a polarity with positive or negative charges, thereby enhancing a dustproof effect.

As a specific aspect, the mask body 100 according to the present invention includes an outer filter and an inner filter, and may be configured to selectively further include a central filter between the outer filter and the inner filter.

It is preferable that the outer filter introduces air introduced from the outer direction in an inner direction, and has a waterproof function such that moisture does not permeate. It is preferable to three-dimensionally to form an air layer through which sufficient air can be introduced between an inner surface of the outer filter and the wearer's face.

It is preferable that the inner filter is combined with the outer filter so as to be located on the inner surface of the outer filter to cover the wearer's nose and mouth, introduces air flowing through the outer filter in the inner direction, and has a waterproof function such that moisture does not permeate.

In addition, the inner filter may be three-dimensionally manufactured to breathe smoothly by dividing into upper and lower portions to form a space between the wearer's nose and mouth such that the breathing of the wearer is comfortable.

If necessary, a nose piece 104 that is deformed to correspond to the shape of the wearer's nose may be provided between the outer filter and the inner filter. The nose piece 104 is deformed to correspond to the shape of the wearer's face such that the inner surface of the inner filter and the wearer's face are in close contact with each other, and then maintains the shape thereof, and may be configured as a wire.

If necessary, the nose piece 104 may be made of the same material as the mask body 100 such that the nose piece 160 may be recycled together with the mask body 100.

The central filter may be configured to have a double structure in which a melt blown filter (MB filter) or an electrostatic filter is stacked in order to improve the filtering ability of the mask body 100.

As described above, when the waterproof function is provided to the inner filter, since moisture contained in the breath is not transferred to the central filter, it is possible to prevent the performance of the electrostatic filter from deteriorating due to moisture.

As an embodiment, the mask body 100 according to the present invention may include a mouth contact sheet that covers the wearer's mouth and nose, a nose contact sheet that is bonded to the upper portion of the mouth contact sheet to cover the wearer's nose, and a chin contact sheet that is bonded to the lower portion of the mouth contact sheet to cover the wearer's chin. If necessary, a nose hanging portion that is curved and sunken to be in contact with the wearer's nose may be formed on the nose contact sheet, and a chin hanging portion that is curved and sunken to be in contact with the wearer's chin may be formed on the chin contact sheet.

The bonding line 140 may be formed in a solid line or a broken line, and may include a first edge bonding line formed between the nose bonding sheet along the edge of the mouth contact sheet and a second edge bonding line formed between the chin contact sheet along the edge of the mouth contact sheet.

The nose contact sheet is in close contact with the rear surface of the mouth contact sheet and then spreads along the upper bonding line when the wearer uses the mask to closely adhere to the nose and wearer's face. The chin contact sheet is in close contact with the rear surface of the mouth contact sheet 102 and then spreads along the upper bonding line when the wearer uses the mask to closely adhere to the wearer's chin and face.

In this way, the mask body 100 according to the present invention may be formed in a structure of a stereoscopic 3D mask including the mouth contact sheet, the nose contact sheet, and the chin contact sheet.

Meanwhile, the mask body 100 according to the present invention may include the contact member.

The contact member is to temporarily reduce the upper and lower lengths of both ends of the mask body 100 such that the left and right ends are not separated from the wearer's face when the mask body 100 is covered on the wearer's face.

Specifically, the contact member includes a first cutting line 110 provided in the upper left end of the mask body 100 to form a first fastening protrusion 115, a first fastening protrusion cutting line 120 provided in the lower left end of the mask body 100 and providing a space in which the first fastening protrusion 115 is to be fitted so as to maintain a state that the upper left end is in close contact with the lower left end, a second cutting line 130 provided in the upper right end of the mask body 100 to form a second fastening protrusion 135 by an external force, and a second fastening protrusion cutting line 140 provided in the lower right end of the mask body 100 and providing a space in which the second fastening protrusion 135 is to be fitted so as to maintain a state that that the upper right end is in close contact with the lower right end.

The first cutting line 110 has an open curve structure such that the first fastening protrusion 115 is not separated while protruding from the mask body 100 by the manipulation of a user. In other words, the first cutting line 110 is omitted in a part in which the first fastening protrusion is connected to the mask body 100.

The first fastening protrusion 115 includes a head portion that is inserted into the first fastening protrusion cutting line to form a hooking projection so as not to be separated, and a body portion connecting the head portion to the mask body 100. The first fastening protrusion cutting line may be formed in any structure as long as the first fastening protrusion 115 may penetrate.

If necessary, the contact member may include a first additional cutting line 150 between the first cutting line 110 and the first fastening protrusion cutting line 120. Such a first additional cutting line 150 is to provide a space which the first fastening protrusion 115 is to penetrate such that the left end of the mask body 100 is in close contact with three layers according to the size of the wearer's face.

The first cutting line 110 and the first fastening protrusion cutting line 120 may provide a function of reducing the vertical length of the left end of the mask body 100.

The second cutting line 130 has an open curve structure such that the second fastening protrusion 135 protrudes and is not separated from the mask body 100 by the manipulation of the user.

If necessary, the contact member may include a second additional cutting line 160 between the second cutting line 130 and the second fastening protrusion cutting line 140.

FIG. 6 is a perspective view showing a mask according to another embodiment of the present invention.

Referring to FIG. 6, the first cutting line 110, the first fastening protrusion cutting line 120, the second cutting line 130, and the second fastening protrusion cutting line 140 may provide an earrings mounting function of selectively attaching/detaching earrings 210 and 220 to/from the mask body 100. To this end, the mask body 100 may include an earrings cutting line 170 to each of an upper portion of the first cutting line 110, a lower portion of the first fastening protrusion cutting line 120, an upper portion of the second cutting line 130, and a lower portion of the second fastening protrusion cutting line 140.

The earrings cutting line 170 temporarily provides a space which the earrings 210 and 220 may penetrate by the external force of a user such that the earrings 210 and 220 may be fixed while penetrating the inside of the mask body 100.

Since an open space is reduced when the supply of the external force from the user is stopped, the earrings cutting line 170 compresses the interpolated earrings 210 and 220 to penetrate the open space and provides pressure such that the earrings 210 and 220 remain coupled to the mask body 100.

Specifically, the earrings cutting line 170 includes a first earrings cutting line provided in the upper portion of the first cutting line 110, a second earrings cutting line provided in the lower portion of the first fastening protrusion cutting line 120, a third earrings cutting line provided in the upper portion of the second cutting line 130, and a fourth earrings cutting line provided in the lower portion of the second fastening protrusion cutting line 140.

Accordingly, the first earring 210 may pass through the first earrings cutting line in the rear surface of the mask body 100 and then pass through the first cutting line 110 in the front surface of the mask body 100 as shown in FIG. 6. Subsequently, the first earring 210 may pass through the first fastening protrusion cutting line 120 in the rear surface of the mask body 100 and then pass through the second earrings cutting line in the front surface of the mask body 10.

The same method is also applied to the second earring 220.

In addition, when the wearer pulls the first earring 210 located between the first cutting line 110 and the first fastening protrusion cutting line 120 in the rear surface of the mask body 100, the wearer may adjust the length of the first earring 210.

As described above, the contact member according to the present invention provides the effect of exchanging only the mask body 100 and reusing the earrings 210 and 220 and the effect that the wearer manually may adjust the lengths of the earrings 210 and 220.

The multi-purpose cutting line is a configuration formed in plural in the bonding surface 102 such that a sheet providing an additional function may be inserted.

As the sheet, a functional pad such as a promotion pad, a humidification pad, an aroma pad, an oxygen pad, a deodorizing pad, etc. may be used. Here, the functional pad means a pad that may be used for a special purpose during daily life or sleeping.

Although the functional pad may be used by interpolating one end to the multi-purpose cutting line, the functional pad may be embedded by including a cutting line sufficient to accommodate the pad in the inner filter or the outer filter.

Referring to FIGS. 3 to 6, the mask according to the present invention includes the earrings 210 and 220.

The first earring 210 and the second earring 220 may be attached to the mask body 100 through a method such as thermal fusion or ultrasonic fusion.

As an example, the earrings 210 and 220 according to the present invention may be formed in an open structure that can be separated from the mask body 100. At this time, one end and the other end of the earrings 210 and 220 may respectively penetrate the earrings cutting line 170, the first cutting line 110, and the first fastening protrusion cutting line 120 provided in the left side of the mask body 100.

If necessary, the earrings 210 and 220 formed in an open curve structure may be formed such that both end portions have a structure thicker than the central portion. This is to prevent the earrings cutting line 170 from penetrating so as not to separate from the mask body 100 after the end portions of the earrings 210 and 220 penetrate the earrings cutting line 170 by an external force of the user, even if an external force pulling the end portions in the direction opposite to the interpolation direction is applied.

Referring to FIG. 6, the mask according to the present invention may further include an earrings connection rod 300.

The earrings connection rod 300 is mounted on the earrings 210 and 220 by a user when using reusable earrings that can be separated from the mask body 100 and converts the earrings to have a closed curve structure by coupling one end of the earrings and the other end of the earrings to each other.

If necessary, the earrings connection rod 300 may be made of a material such as silicone, rubber, or plastic foam. This is to relieve pain in the ear when the earrings connection rod 300 comes into contact with the user's ear, and, even if a pulling force is applied to the interpolated earring, to prevent separation from the earrings connection rod 300 by generating a frictional force with the earrings and the earrings connection rod 300.

The earrings connection rod 300 may be formed in any structure as long as the earrings connection rod 300 is coupled to one end and the other end of the earrings formed in an open curve structure to form the earrings formed in the open curve structure as that of a closed curve structure.

As a first embodiment, the earrings connection rod 300 according to the present invention may be formed in a clip structure that presses together one end of the earrings and the other end of the earrings to connect the entire earrings in a closed curve structure.

As a second embodiment, the earrings connection rod 300 according to the present invention may be formed in a ring-shaped structure or a rod-shaped structure that presses together one end of the earrings and the other end of the earrings to connect the entire earrings in a closed curve structure.

Here, the rod-shaped structure may be formed in a ‘U’ shape such that the earrings connection rod 300 may be smoothly wound around the user's ear. At this time, one end of the earrings 210 and 220 are interpolated into the front end of the earrings connection rod 300, and the other end of the earrings 210 and 220 is interpolated into the rear end of the earrings connection rod 300. To this end, the earrings connection rod 300 has an interpolation passage through which the earrings 210 and 220 may be inserted in the longitudinal direction.

In addition, an opening through which the earrings are exposed to the outside may be provided in the interpolation passage of the earrings connection rod 300. This is to expose one end of the earrings and the other end of the earrings penetrating the interpolation passage to the outside of the earrings connection rod 300. At this time, the wearer may adjust the length of the earrings wrapping the wearer's ear by pulling the end of the earrings exposed to the outside of the earrings connection rod 300.

In addition, the interpolation passage may be formed to have an inner diameter smaller than an outer dimeter of the earrings 210 and 220 such that the earrings 210 and 220 may not be separated from the insertion passage of the earrings connection rod 300 even if a pulling force is applied to the earrings after the ends of the earrings 210 and 220 are interpolated. In particular, the inner diameter of the tip to which the ends of the earrings 210 and 220 enter may be the narrowest, and the inner diameter may be formed to gradually increase in the longitudinal direction.

In addition, the earrings connection rod 300 may include an earring interpolation cutting line connected to the interpolation passage along the longitudinal direction such that the ends of the earrings 210 and 220 may be easily interpolated into the interpolation passage. At this time, the earring interpolation cutting line is preferably provided in a direction opposite to the surface facing the mask body 100 as shown in FIG. 6 such that the earrings are not separated from the earrings connection rod 300 by the pulling force applied to the earrings.

As a third embodiment, the earrings connection rod 300 according to the present invention may include a first earrings connection rod and a second earrings connection rod that may be assembled with or separated from each other.

The first earrings connection rod includes a first interpolation passage through which one end of the earrings may be interpolated along the longitudinal direction such that the entire length of the earrings of the mask may be adjusted, a first cutting line connected to the first interpolation passage along the longitudinal direction such that one end of the earrings is interpolated into the first interpolation passage, and a coupling protrusion on the end. In addition, the first earrings connection rod may include a first opening connected to the first interpolation passage in the surface such that one end of the earrings may be disposed outside after penetrating the first earrings connection rod.

The second earrings connection rod includes a second interpolation passage through which the other end of the earrings may be interpolated along the longitudinal direction such that the entire length of the earrings of the mask may be adjusted, a second cutting line connected to the second interpolation passage along the longitudinal direction such that the other end of the earrings is interpolated into the second interpolation passage, and a coupling grove assembled with and connected to the coupling protrusion in the end facing the first earrings connection rod. In addition, the second earrings connection rod may include a second opening connected to the second interpolation passage in the surface such that the other end of the earrings may be disposed outside after penetrating the second earrings connection rod.

Such a coupling protrusion may be formed in a cylinder, a square pillar, a square pillar, a pentagonal pillar, etc. such that the coupling protrusion may be interpolated and coupled to the coupling groove, and the coupling groove may be also formed in a structure corresponding to the coupling protrusion such that the coupling protrusion may be interpolated.

In particular, when the coupling protrusion is formed in a cylindrical structure, a screw thread may be formed on the surface of the coupling protrusion for a firm coupling with the coupling groove, and a screw groove may be formed in the coupling groove.

In this way, the earrings connection rod 300 may be used as an ear pain protector, and may also provide an earrings adjustment function because the earrings may be pulled and fixed.

While the present invention has been described with reference to the particular illustrative embodiments, it is not to be restricted by the embodiments but only by the appended claims. It is to be appreciated that those skilled in the art may change or modify the embodiments without departing from the scope and spirit of the present invention.

What is claimed is:

1. A mask comprising a mask body that covers wearers' mouth and nose, and earrings respectively provided on left and right sides of the mask body, wherein the mask body comprises a contact member comprising a first cutting line provided in the upper left end of the mask body to form a first fastening protrusion by an external force, a first fastening protrusion cutting line provided in the lower left end of the mask body and providing a space in which the first fastening protrusion is to be fitted so as to maintain a state that the upper end is in close contact with the lower end, a second cutting line provided in the upper right end of the mask body to form a second fastening protrusion by an external force, and a second fastening protrusion cutting line provided in the lower right end of the mask body and providing a space in which the second fastening protrusion is to be fitted so as to maintain a state that that the upper right end is in close contact with the lower right end.

2. The mask according to claim 1, wherein the first and second fastening protrusions include a head portion that is inserted into the first and second fastening protrusion cutting lines to form a hooking projection so as not to be separated, and a body portion connecting the head portion to the mask body.

3. The mask according to claim 2, wherein the head portion is formed in any one of an arrow shape, a triangle, a square, a pentagon, a hexagon, and a circle, and wherein the first and second fastening protrusion cutting lines are formed in any one of a ‘ [symbol] ’ shape, a ‘ [symbol] ’ shape, a ‘\hat{ }’, a ‘ [symbol] ’ shape, and a ‘ [symbol] ’ shape.

4. The mask according to claim 1, wherein the contact member includes a first additional cutting line providing a space which the first fastening protrusion is to be fitted between the first cutting line and the first fastening protrusion cutting line; and
a second additional cutting line providing a space which the second fastening protrusion is to be fitted between the second cutting line and the second fastening protrusion cutting line.

5. The mask according to claim 1, wherein the mask body comprises a receiving hole in an inner surface or an outer surface of the mask body such that a sheet is fitted therein, or a plurality of multi-purpose cutting lines in the bonding surface of the mask body.

6. The mask according to claim 5, wherein the sheet is any one of a promotion pad, a humidification pad, an aroma pad, an oxygen pad, a deodorizing pad.
